(19) United States
(12) Patent Application Publication
(10) Pub. No.: US 2007/0260256 A1
(43) Pub. Date: Nov. 8, 2007

(54) SURGICAL INSTRUMENT TRAY, HIP RESURFACING KIT, AND METHOD OF RESURFACING A FEMORAL HEAD TO PRESERVE FEMORAL HEAD VASCULARITY

(76) Inventor: Paul E. Beaule, Ottawa (CA)

Correspondence Address: MATTHEW M. ROY, 2241 DES GRANDS CHAMPS STREET, OTTAWA, ON, K1W 1K1, US

(21) Appl. No.: 11/742,864

(22) Filed: May 1, 2007

Related U.S. Application Data
(60) Provisional application No. 60/746,504, filed on May 5, 2006.

(51) Int. Cl. A61B 17/16; A61B 17/88

(52) U.S. Cl. 606/80; 606/84; 606/102; 623/908

(57) ABSTRACT

A method of resurfacing a femoral head involves using a chamfered reamer to ream only a top portion of the femoral head above an anterior lateral portion of the femoral head where the retinacular vessels supply blood to the femoral head. As no cylindrical reaming is performed on the lateral portions of the femoral head, damage to the retinacular vessels is avoided. The method thereby preserves femoral head vascularity. A surgical instrument tray for enabling a surgeon to perform this method includes both a reamer and a spherometer for assessing the sphericity of the femoral head to determine whether osteophyte growth on the femoral head is likely to cause femoro acetabular impingement. The instrument tray can also include a tool, such as a bone chisel or burr, for removing the osteophytes to restore the sphericity of the femoral head, rather than removing the osteophytes using a cylindrical reamer.

### CROSS-REFERENCE TO RELATED APPLICATIONS

This application claims priority under 35 U.S.C. §119(e) from U.S. Provisional Patent Application Ser. No. 60/746,504 filed on May 5, 2006.

### FIELD OF THE INVENTION

The present invention relates generally to orthopedic surgery and, in particular, to hip resurfacing.

### BACKGROUND OF THE INVENTION

A hip joint may degenerate due to a variety of etiologies, often necessitating replacement of the natural hip joint with a prosthetic hip. In a conventional hip replacement (or hip arthroplasty), the femoral head is removed altogether and replaced with a prosthetic femoral insert having a stem and ball-shaped cup as is well known in the art. Furthermore, in a conventional hip replacement, the head of the femur is resected to create a cavity within the intramedullary canal of the femur for accepting the hip prosthesis, i.e. the femoral insert. A conventional hip replacement can be a total or partial arthroplasty. In the latter case, also known as a hemiarthroplasty, only the femoral side is replaced, leaving the implanted femoral component to articulate within the acetabulum's natural socket). In a total hip arthroplasty, i.e. a total hip replacement, the hip socket (acetabulum) is also replaced with an acetabular component (resulting, for example, in a metal-to-metal ball-and-socket joint).

Both the partial and total conventional hip replacements are considered to be fairly drastic surgery, requiring removal not only of the femoral head (and a portion of the femoral neck) but also of a significant amount of bone tissue from the area surrounding the proximal intramedullary canal. Moreover, in certain cases, the stem of the femoral implant can loosen within the femur over the years, especially in younger, active patients, resulting in failure, pain and thus requiring surgical revisions.

An alternative to the conventional hip replacement is a hip resurfacing, which is much less invasive than the conventional hip arthroplasty. A hip resurfacing is indicated for those patients who still have sufficient bone stock, i.e. where the diseased region of the femoral head is confined to the surface of the femoral head, and the subsurface bone tissue is essentially healthy. As is known in the art, slight-to-moderate bone tissue damage on the surface of the femoral head may arise due to a vascular necrosis (osteonecrosis), bony dysmorphisms, dysplasia, traumatic causes, or metabolic causes which, in certain cases, leaves much of the femoral neck and femoral head in a fairly healthy state. In other words, the persistence of subsurface bone stock contraindicates the removal of the entire femoral head to accommodate a conventional femoral insert. In those cases, a resurfacing preserves the maximum amount of bone tissue as well as the integrity of the intramedullary canal.

Therefore, implanting femoral resurfacing components requires only that a portion of the femoral head be resected, rather than the entire femoral head. The amount of bone which must be resected in a hip resurfacing is much less since the femoral head component does not have a long stem as it does in a conventional hip replacement. Unlike the conventional hip replacement arthroplasty, where all of the femoral head is replaced with a prosthetic implant, hip resurfacing entails replacing the outer articulating portion of the femoral head with a much thinner implant. Like the conventional hip replacement, a hip resurfacing procedure may be limited to a hemiarthroplasty, where only the femoral side of the hip is replaced. Alternatively, the hip resurfacing may be a total joint anthroplasty in which both femoral and acetabular sides of the joint are replaced with a matching acetabular implant that fits within the hip socket or acetabulum.

FIG. 1 illustrates a femoral head and neck with a cutaway of the superolateral head-neck junction showing the retinacular vessels in the anterior lateral portion of the femoral head-neck region. As shown in FIG. 1, the femoral head and neck, which is generally designated by reference numeral 10, has a femoral neck 12 connected to a femoral head 14 to form a ball-and-socket joint with the acetabulum. An anterior lateral portion 16 of the femoral head-neck region has a plurality of nutrient retinacular vessels 18 that supply blood to, and thus nourish, the bone tissue of the femoral head.

FIGS. 2 to 4 illustrate a prior-art method of preparing a femoral head 14 for a resurfacing operation. First, as shown in FIG. 2, the femoral head 14 is cylindrically reamed using a cylindrical reamer 20. This cylindrical reaming removes any lateral osteophyte growth 22 and prepares the sides of the femoral head for receiving a femoral head component. Then, as shown in FIG. 3, the top portion 24 of the femoral head 14 is reamed with a chamfered reamer 26 to complete the preparation of the femoral head. A femoral head component 28 is then secured to the prepared top surface 32 of the femoral head 14, as shown in FIG. 4. However, as will be elaborated below, this technique can easily damage the retinacular vessels 18 in the femoral head, thus making the femoral head prone to osteonecrosis, resulting in possible fracture or loosening.

Hip resurfacing technology has evolved in recent years, as evidenced by the following patent references, which are described briefly below.

U.S. Patent Application US 2005/0033447 A1 (Evans) entitled FEMORAL HEAD RESURFACING APPARATUS AND METHODS, which is hereby incorporated by reference, describes a femoral head resurfacing apparatus that includes a hemispherical cap with a stabilization structure of non-shear fixation bars that are inserted into slots cut into the femoral head.

U.S. Pat. No. 4,976,740 (Kleiner) entitled ANCHORED FEMORAL DOME, which is hereby incorporated by reference, describes a femoral head implant having a prepared channel through which a compression bolt is inserted to affix the implant against a sculpted femoral head. The implant has a dome defining a hemispherical bearing surface while the underside of the dome includes downwardly protruding spikes for holding the dome to the sculpted femoral head.

U.S. Pat. No. 6,096,084 (Townley) entitled MODULAR BALL AND SOCKET JOINT PREFERABLY WITH A CERAMIC HEAD BALL, which is hereby incorporated by reference, describes a cupped ball head for use in resurfacing arthroplasty where the cupped ball head has a bore for frictionally engaging an upper, tapered cap of the elongated stem of the implant. The stem can thus be pressed into engagement with the cupped ball head by forcing the tapered cap of the stem through the bore of the ball.

U.S. Patent Application Publication US 2003/0163202 A1 (Lakin) entitled MODULAR RESURFACING PROSTHETIC, which is hereby incorporated by reference, describes a modular femoral head resurfacing system that provides variously configured head and stem components that allows for various angles and offsets to be achieved.

U.S. Pat. No. 4,846,841 (Oh) entitled FEMORAL PROSTHESIS, which is hereby incorporated by reference, describes a femoral head prosthesis including a femoral insert that has a cavity for at least partially a remnant of a femoral head. The insert is affixed to the remnant of the head without cement, and the shell is mounted on the insert by a taper lock. With this invention, few sizes of the femoral shell need to be provided, and cementing can be eliminated.

European Patent Application EP 1 570 811 A1 (Ganz) entitled in German FEMURKOPFPROTHESE (which translates as “Femur Head Prosthesis”), which is hereby incorporated by reference, describes a femoral head component having a spherical cap and a bone-penetrating screw. The spherical cap has a recessed portion designed to be aligned with the ramus acetabularis (arteria circumflexa femoris medialis or acetabular branch of the medial femoral circumflex artery) so that the latter artery need not be removed to accommodate the prosthesis.

One enduring problem with known hip resurfacing techniques is that the use of a cylindrical reamer in the preparation of the femoral head (to receive a femoral head prosthetic) as well as in the restoration of femoral head sphericity (by removing osteophytes) tends to impair the vascularity of the femoral head. Reaming of the lateral portions of the femoral head can damage the retinacular vessels, resulting in diminished blood supply to the femoral head. As a consequence, the bone in the immediate vicinity of the femoral prosthesis tends to degenerate or die (osteonecrosis), often resulting in failure of the prosthetic joint.

Accordingly, it would be highly desirable to develop a technique that would preserve vascularity in the femoral head.

### SUMMARY OF THE INVENTION

An object of the present invention is to provide a method, instrument tray and kit for enabling an orthopedic surgeon to perform a hip resurfacing operation that prepares the femoral head in such a manner that damage to the retinacular vessels is minimized to thus optimally preserve femoral head vascularity. In one embodiment, the femoral head is prepared using a chamfered reamer to ream only a top portion of the femoral head above an anterior lateral portion of the femoral head where the retinacular vessels supply blood to the femoral head. As no cylindrical reaming is performed on the lateral portions of the femoral head, damage to the retinacular vessels is avoided or at least substantially minimized. In another embodiment, the retinacular vessels are avoided by using, for example, an oversized reamer on the femoral head. The method thereby preserves femoral head vascularity which is instrumental in promoting bone tissue survival in the vicinity of the prosthesis. By improving vascularity in the femoral head, the prosthesis or implant is more likely to remain securely affixed to the patient's bone.

A novel surgical instrument tray and a hip replacement kit each includes a reamer and a spherometer for performing a hip resurfacing operation in which the surgeon prepares the femoral head and restores the head's sphericity while preserving femoral head vascularity.

The surgical instrument tray enables an orthopedic surgeon to prepare the femoral head and also, in the same surgery, to remove osteophytes for restoring the sphericity of the femoral head. The surgical instrument tray includes both a reamer for preparing the head and a spherometer for assessing the sphericity of the femoral head to determine whether osteophyte growth on the femoral head is likely to cause femoro acetabular impingement. The instrument tray can also include an osteophyte-removing tool, such as a bone chisel or burr, for removing the osteophytes to restore the sphericity of the femoral head. Removing the osteophytes using a bone chisel and mallet or, alternatively, using a burr is much better than using a cylindrical reamer, as is done conventionally, because the cylindrical reamer tends to damage the retinacular vessels because it engages the whole circumference of the head-neck junction, thus causing diminished femoral head vascularity, leading to osteonecrosis in the vicinity of the prosthesis and, consequently, poor long-term viability of the prosthetic joint.

The hip replacement kit includes a reamer, a spherometer, and a separately packaged femoral head prosthetic component for enabling a surgeon to perform a partial hip resurfacing. The hip replacement kit can also include an acetabular component (also separately packaged) for enabling a surgeon to perform a total hip resurfacing. The hip replacement kit can further include one or more osteophyte-removing tools for correcting the asphericity of the femoral head in the same surgery.

In summary, therefore, the method, instrument tray and kit enables an orthopedic surgeon to preserve the vascularity of the femoral head during preparation of the femoral head for a hip resurfacing operation. The surgeon reams only a top portion of the femoral head, i.e. a portion of the femoral head which is above the retinacular vessels, thus minimizing damage to the area around the retinacular vessels of the femoral head, or alternatively reams around the retinacular vessels using, for example, an oversized reamer (to thus avoid damaging the retinacular vessels). By preserving blood flow into the femoral head, bone tissue is substantially more likely to survive, which results in superior longevity of the prosthetic joint.

Accordingly, one aspect of the present invention provides a method of resurfacing a femoral head to preserve vascularity in the femoral head. The method includes steps of preparing the femoral head by cutting only a top portion of the femoral head, the top portion of the femoral head being above an anterior lateral portion of the femoral head where the retinacular vessels supply blood to the femoral head, and securing a femoral head prosthesis to the femoral head. By only reaming the top portion of the femoral head, the surgeon avoids damaging the retinacular vessels.

Another aspect of the present invention provides a surgical instrument tray for resurfacing a femoral head while preserving vascularity in the femoral head. The instrument tray includes a spherometer for assessing a sphericity of the femoral head, and a reamer for preparing the femoral head. Combining both a reamer and a spherometer into a tray (or as a simple kit, i.e. without the physical tray) is useful for surgery where the patient's femoral head needs to be both prepared (reamed) and restored (in terms of sphericity).

Yet another aspect of the present invention provides a hip resurfacing kit having a spherometer for assessing a sphericity of the femoral head and a reamer for preparing the femoral head to receive a femoral head prosthetic component. The kit can include a femoral head prosthetic component for partial hip resurfacing, or it can include both the femoral head component and the acetabular component for a total hip resurfacing.

Yet a further aspect of the present invention provides a method of resurfacing a femoral head to preserve vascularity in the femoral head where the method includes steps of preparing the femoral head by reaming the femoral head so as to avoid damaging a plurality of retinacular vessels supplying blood to the femoral head and securing a femoral head prosthesis to the femoral head. In one embodiment, this method entails using an oversized reamer to avoid cutting on the anterior lateral portion of the femoral head where the retinacular vessels are situated.

### BRIEF DESCRIPTION OF THE DRAWINGS

Further features and advantages of the present invention will become apparent from the following detailed description, taken in combination with the appended drawings, in which:

FIG. 1 illustrates a femoral head and neck with a cutaway of the superolateral head-neck junction showing the retinacular vessels in the anterior lateral portion of the femoral head-neck region;

FIG. 2 illustrates a method of cylindrically reaming a femoral head having osteophyte growth in accordance with the prior art;

FIG. 3 illustrates a method of preparing the femoral head using a chamfered reamer, after having cylindrically reamed the femoral head as shown in FIG. 2, also in accordance with the prior art;

FIG. 4 illustrates a method of affixing a femoral head prosthesis to the prepared head in accordance with the prior art;

FIG. 5 illustrates the assessment and restoration of the sphericity of a femoral head, which is performed as part of a method in accordance with an embodiment of the present invention;

FIG. 6 illustrates the preparation of the femoral head using a chamfered reamer, which is performed as part of the method in accordance with an embodiment of the present invention;

FIG. 7 illustrates the affixing of a femoral head component to a prepared top portion of a femoral head, which is performed as part of the method in accordance with the embodiment of the present invention;

FIG. 8 is an enlarged view of the femoral head and neck, illustrating how a chamfered reamer prepares the femoral head above the retinacular vessels;

FIG. 9 is an isometric perspective view of a spherometer for use in a surgical instrument tray and/or in a hip resurfacing kit in accordance with embodiments of the present invention;

FIG. 10A is an isometric perspective view of a chamfered reamer for use in a surgical instrument tray and/or in a hip resurfacing kit in accordance with embodiments of the present invention;

FIG. 10B is a cross-sectional view of the chamfered reamer of FIG. 10A;

FIG. 11 is an isometric perspective view of a surgical instrument tray having a reamer and a spherometer in accordance with an embodiment of the present invention;

FIG. 12 is a schematic top view of a surgical instrument tray having a reamer and a spherometer in accordance with an embodiment of the present invention;

FIG. 13 is a schematic top view of a surgical instrument tray having differently sized reamers and spherometers in accordance with another embodiment of the present invention;

FIG. 14 is a schematic top view of a surgical instrument tray having a reamer, a spherometer, a burr, a chisel and a mallet in accordance with another embodiment of the present invention;

FIG. 15 is a schematic top view of a surgical instrument tray having differently sized reamers, spherometers, burrs and chisels in accordance with an embodiment of the present invention;

FIG. 16 is a schematic top view of a hip resurfacing kit having a reamer and a spherometer and a separately packaged femoral head prosthesis in accordance with another embodiment of the present invention;

FIG. 17 is a schematic top view of a hip resurfacing kit having a reamer and a spherometer along with separately packaged femoral head and acetabular prostheses in accordance with another embodiment of the present invention; and

FIG. 18 is a schematic top view of a hip resurfacing kit having a reamer, a spherometer, a burr, a chisel and a mallet along with separately packaged femoral head and acetabular prostheses in accordance with another embodiment of the present invention.

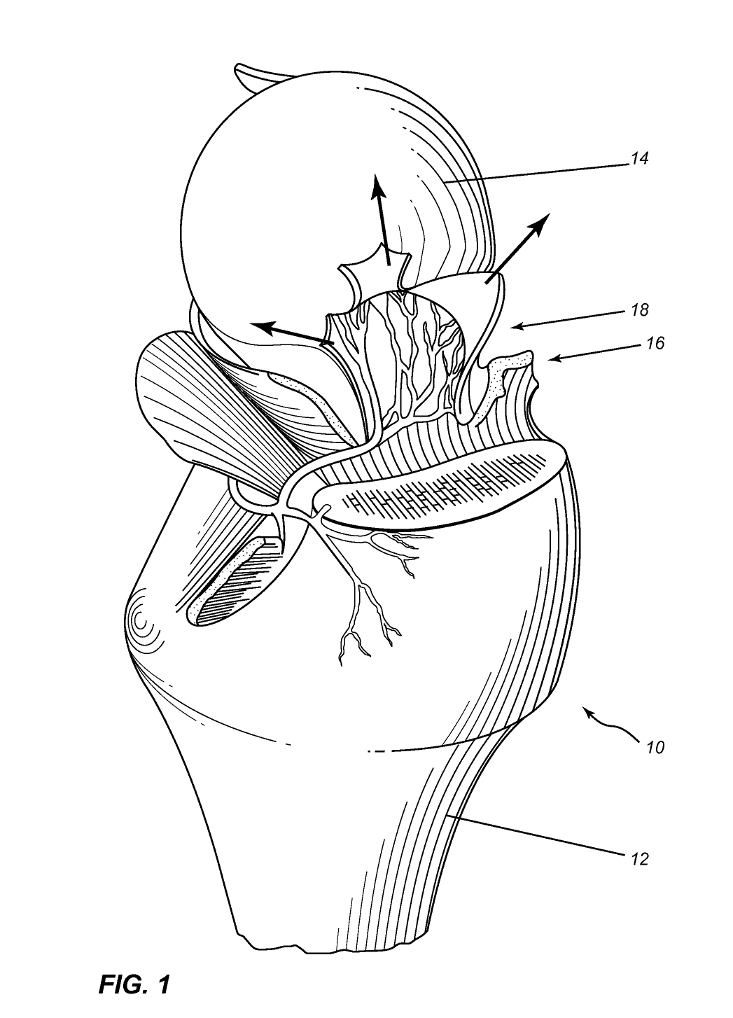

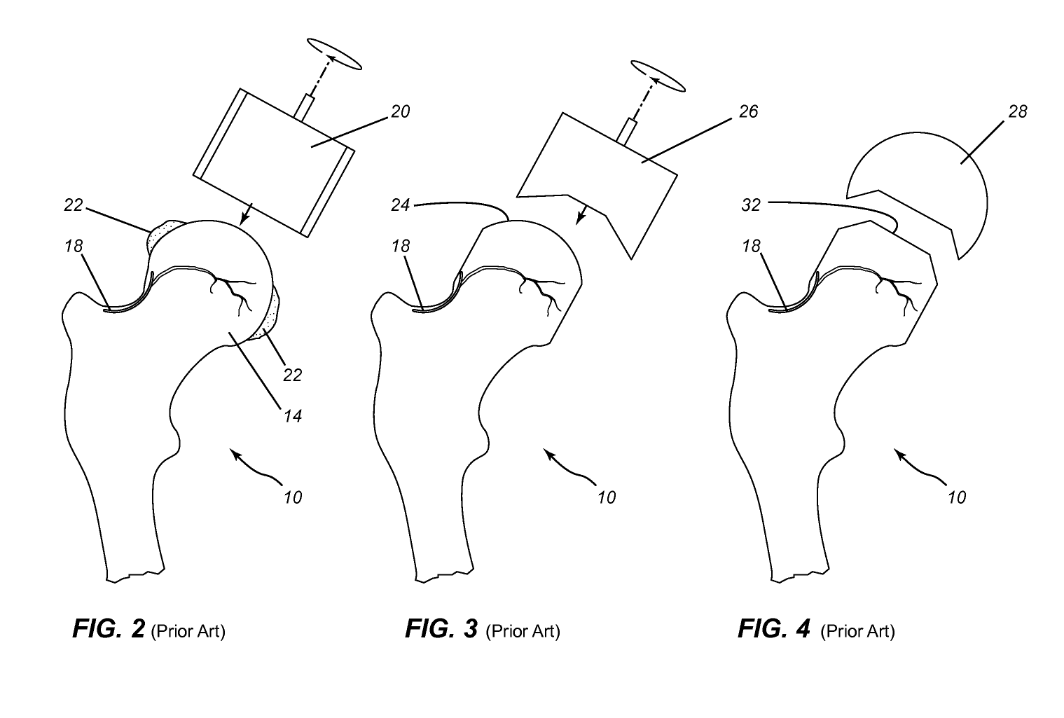

### DETAILED DESCRIPTION OF THE PREFERRED EMBODIMENTS

A method of resurfacing a femoral head to preserve vascularity in the femoral head in accordance with a preferred embodiment of the present invention includes a step of preparing the femoral head by cutting (e.g. reaming) only a top portion of the femoral head, preferably using a chamfered reamer. The “top portion” of the femoral head shall be understood in the present application to mean the portion of the femoral head that is above an anterior lateral portion of the femoral head where the retinacular vessels supply blood to the femoral head, as illustrated in the accompanying figures. The method also includes a step of securing a femoral head prosthesis to the femoral head once the femoral head has been prepared in this vascular-preserving manner. In another embodiment, the method avoids the retinacular vessels by reaming around these vessels, for example, by using an oversized reamer, in which case, it would be possible to cut beneath the top portion.

In addition, the method can further include steps of restoring the sphericity of the femoral head, such as in the case where osteophytes (also known as “bone spurs”) have grown on the femoral head, usually due to arthritis, causing the femoral head to lose its sphericity, i.e. to become aspherical. As is known in the art, osteophytes are outgrowths or protuberances of bone occurring at an osteoarthritic joint that usually limit joint movement and cause pain due to femoro acetabular impingement whereby the aspherical ball of the femur (i.e. the osteophyte protruding from the sides of the femoral head) impinges on the acetabulum when the femoral head rotates within the socket of the acetabulum. Where osteophtye growth causes a femoral head to become sufficiently aspherical that femoro acetabular impingement occurs, the sphericity of the ball of the femur needs to be corrected. Part of the resurfacing operation can thus involve restoring the sphericity of the patient's femoral head (i.e. in addition to preparing the femoral head and implanting a femoral head prosthetic component). Thus, in addition to preparing the femoral head by reaming a top portion above the retinacular vessels, the method can also include steps of (i) assessing a sphericity of the femoral head to determine whether osteophyte growth on the femoral head is likely to cause femoro acetabular impingement and (ii) removing osteophytes from the femoral head to restore the sphericity of the femoral head. Assessing a sphericity of the femoral head can be performed using a spherometer. Preferably, as shown in the figures, the spherometer is a hand-held device having a handle for enabling the surgeon to hold the spherometer and having a concave spherical gauge connected to the handle. The concave spherical gauge is adapted to contact an outer surface of the femoral head to enable an orthopedic surgeon to assess the sphericity of the femoral head. The concave spherical gauge has a curvature matching that of a femoral head. The spherometer also permits optimization of the femoral head-neck ratio, or of the head-neck offset. This gives better visualization of the true axis of the femoral neck which minimizes the risk of notching and permits accurate implant sizing. A set of spherometers can be provided, each having spherical gauges of slightly different curvature for use on patients whose femoral heads are of different sizes/curvatures.

Removing osteophytes can be accomplished using an osteophyte-removing tool such as a bone chisel or burr (which are tools already well known in the art) instead of using a cylindrical reamer (as is often done in the prior art). Use of a cylindrical reamer can cause damage to the anterior lateral portion of the femoral head where the retinacular vessels supply blood to the femoral head. In accordance with this novel method, the orthopedic surgeon would use a bone chisel, burr or other such tool to carefully remove the osteophytes from the femoral head without damaging the retinacular vessels.

By using the spherometer and osteophyte-removing tool, the orthopedic surgeon can assess and restore the sphericity of the femoral head without having to cylindrically ream the femoral head, which tends to damage the retinacular vessels, causing osteonecrosis and poor longevity of the prosthesis.

In accordance with another aspect of the present invention, a surgical instrument tray is provided for enabling an orthopedic surgeon to implement the foregoing method, i.e. to resurface a femoral head while preserving vascularity in the femoral head. The surgical instrument tray contains the instruments that an orthopedic surgeon would need to implement the foregoing method, i.e. the tools the orthopedic surgeon would need to prepare the femoral head by cutting only a top portion of the femoral head, i.e. the portion of the femoral head above an anterior lateral portion of the femoral head where the retinacular vessels supply blood to the femoral head. The instrument tray thus includes a reamer for preparing the femoral head in a vascular-preserving manner. Preferably, the reamer is a chamfered reamer for preparing only a top portion of the femoral head that is situated above an anterior lateral portion of the femoral head where the retinacular vessels supply blood to the femoral head.

In addition to the reamer for preparing the femoral head, the surgical instrument tray also includes a spherometer for assessing a sphericity of the femoral head. As noted above, the spherometer preferably has a handle for holding the spherometer and a concave spherical gauge connected to the handle, the concave spherical gauge being adapted to contact an outer surface of the femoral head to enable an orthopedic surgeon to assess the sphericity of the femoral head.

Preferably, the surgical instrument tray further includes a tool for removing osteophytes from the femoral head to enable an orthopedic surgeon to restore the sphericity of the femoral head to minimize femoro acetabular impingement when the femoral head rotates relative to the acetabulum. The tool is preferably selected from the group consisting of bone chisels and burrs, although similar such tools can be utilized, as will be appreciated by those of ordinary skill in the art of orthopedic surgery, provided they do not damage the retinacular vessels the way that a cylindrical reamer is likely to do. With the spherometer and osteophyte-removing tool, the orthopedic surgeon can restore the sphericity of the femoral head. As idiopathic osteoarthritis is the most common etiology treated with hip resurfacing, it would be highly useful to provide a surgical instrument tray having a spherometer and an osteophyte-removing tool (for correcting the asphericity) and a chamfered reamer for preparing the femoral head.

For the purposes of the present specification, it should be understood that the surgical instrument tray is a container, tray or other device that contains a reamer and a spherometer for performing this novel vascular-preserving resurfacing operation. The reamer and spherometer could also be provided together as a pair of instruments/tools without any container, tray or holding device.

In accordance with yet another aspect of the present invention, a hip resurfacing kit includes not only the tools of the surgical instrument tray (i.e. the reamer for preparing the femoral head to receive the femoral head prosthetic component and the spherometer for assessing a sphericity of the femoral head) but further includes a femoral head prosthetic component (femoral implant or femoral insert). The femoral implant can be any type of femoral implant, i.e. implants that are bonded using bone cement, implants secured using threaded fasteners, implants having stems that penetrate into the intramedullary canal of the femur, or implants using biological fixation, i.e. cementless. The kit, however, could also be simply the reamer and the spherometer (without any prosthesis) which is thus effectively the surgical instrument tray minus the tray itself or minus whatever element physically holds or packages the instruments.

In another embodiment, the hip resurfacing kit can further include an acetabular prosthetic component for attaching to an acetabulum. Thus, the hip resurfacing kit can be provided in various forms, one for a partial (or hemi) resurfacing operation (i.e. femoral side only) and the other for a full resurfacing (i.e. both the femoral side and the acetabular side).

Furthermore, the hip resurfacing kit can further include a tool for removing osteophytes from the femoral head. As was noted above, the tool is preferably selected from the group consisting of bone chisels and burrs, although other non-damaging tools can be used.

The reamer in the hip resurfacing kit is preferably a chamfered reamer which enables the orthopedic surgeon to ream only a top portion of the femoral head that is situated above an anterior lateral portion of the femoral head to thereby avoid damaging the retinacular vessels in the anterior lateral portion of the femoral head. Alternatively, the reamer could be a spherical reamer, or a reamer having cutters designed to cut any other profile provided it does not cut into the region where the retinacular vessels are located.

The method, surgical instrument tray and resurfacing kit described above help to reduce the likelihood of failure of an implanted femoral component. Extra-osseous blood is believed to be a significant contributor to femoral head blood flow as validated by laser doppler flowmetry of the anterior lateral portion of the femoral head. Accordingly, osteonecrosis still represents a potential contributor to implant failure. Damage to the retinacular vessels thus constitutes an osteonecrotic event which increases the risk of femoral loosening during the repair phase. Therefore, the embodiments of the present invention are believed to improve the survivability of hip resurfacing implants by preserving vascularity in the femoral head. By preserving vascularity, this would enhance biological (cementless) fixation.

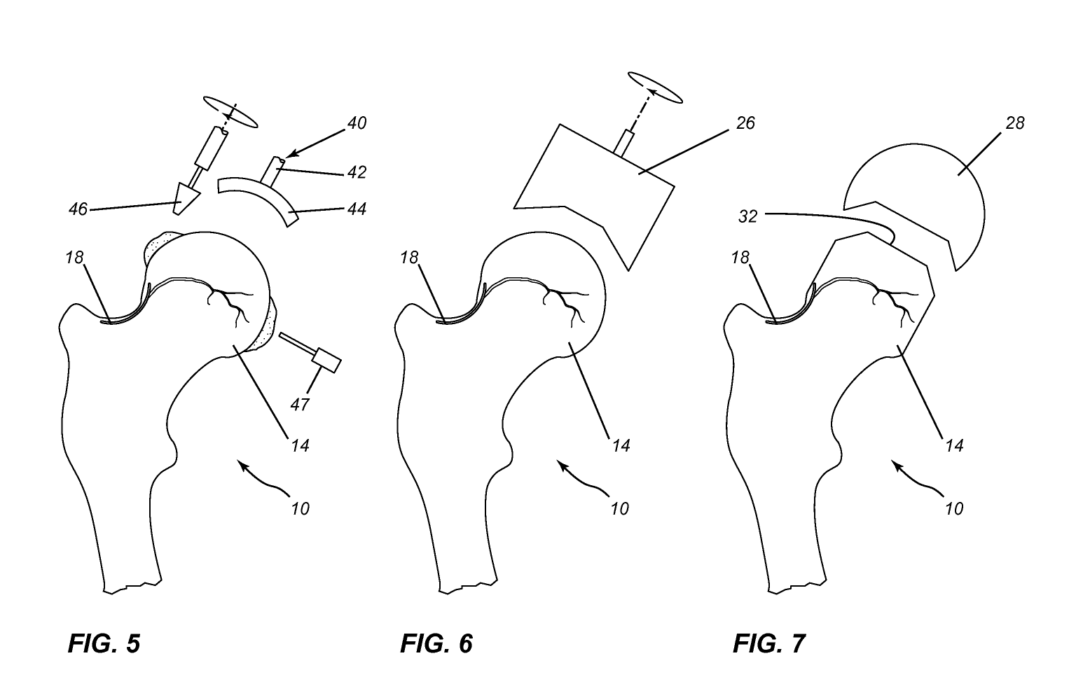

FIGS. 5 to 7 illustrate the novel method as well as the use of the novel surgical instrument tray and kit described above. As shown in FIG. 5, a spherometer 40 having a handle 42 and a concave spherical gauge 44 is run over the surface of the femoral head to assess its sphericity. If osteophyte growth has caused asphericity, the sphericity is then restored using an osteophyte-removing tool such as the burr 46 (or bone chisel 47) shown in FIG. 5 (or by using an osteotome or other appropriate tool). The surgeon removes the osteophytes while intermittently checking the sphericity until he or she is satisfied that the femoral head has the desired sphericity.

As shown in FIG. 6, a reamer 26 (which is preferably a chamfered reamer or alternatively a spherical reamer) is then used to prepare the femoral head 14. The chamfered reamer 26 is used to prepare/cut the top portion of the femoral head above the retinacular vessels 18 to ensure that no damage (or at most only very minimal damage) is done to the retinacular vessels 18 to preserve the vascularity of the femoral head.

As shown in FIG. 7, once the femoral head 14 has been prepared, the femoral head component 28 (femoral head implant) can then be affixed (by any known technique, e.g. cementing, threaded fastening, etc.) to the prepared top surface 32 of the femoral head.

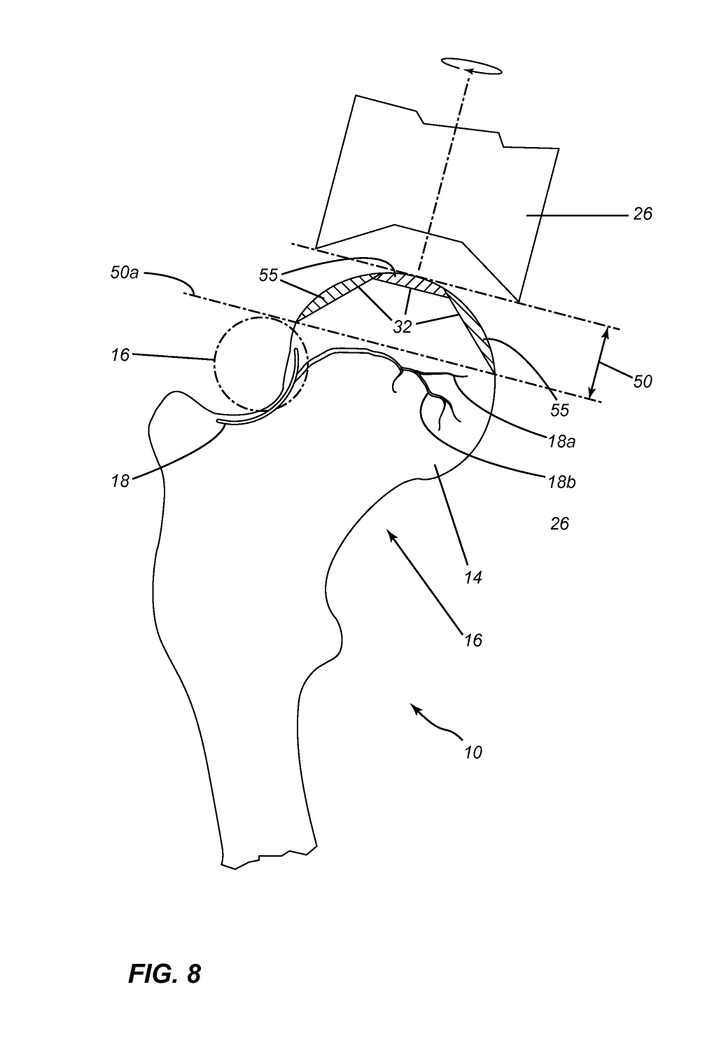

For greater clarity, FIG. 8 shows an enlarged view of the femoral head and neck 10. The anterior lateral portion 16 of the femoral head (which is also referred to herein as the anterior lateral portion of the head-neck region) is the vulnerable portion containing the nutrient retinacular vessels 18 that must not be damaged during preparation/reaming of the femoral head or during osteophyte removal. The chamfered reamer is used to cut away portions 55 of the top surface of the femoral head, leaving behind a prepared femoral head 32. For the purposes of this specification, “the top portion of the femoral head” shall mean the portion identified by reference numeral 50, i.e. the “top portion” 50 above an anterior lateral portion 16 of the femoral head where the retinacular vessels 18 (and its downstream branches 18a and 18b) supply blood to the femoral head.

Therefore, the best mode known to the inventor at the time of filing this application is to prepare the femoral head where the only reamer used is a chamfer reamer, i.e. without first using a cylindrical reamer. Although the best mode is to use a chamfer reamer to ream above the line 50a delineating the “top portion” 50 as shown in FIG. 8, it is also possible, in a variant of this method, to use an oversized reamer to ream below line 50a such that the oversized reamer does not cut into the anterior lateral portion 16 where the retinacular vessels 18 are situated. This alternative method would therefore enable a surgeon to ream the femoral head while avoiding the retinacular vessels. In this variant, however, as will be appreciated by those of skill in the art of orthopedic surgery, a number of issues arise for installing the component after having made this sort of “asymmetrical” cut.

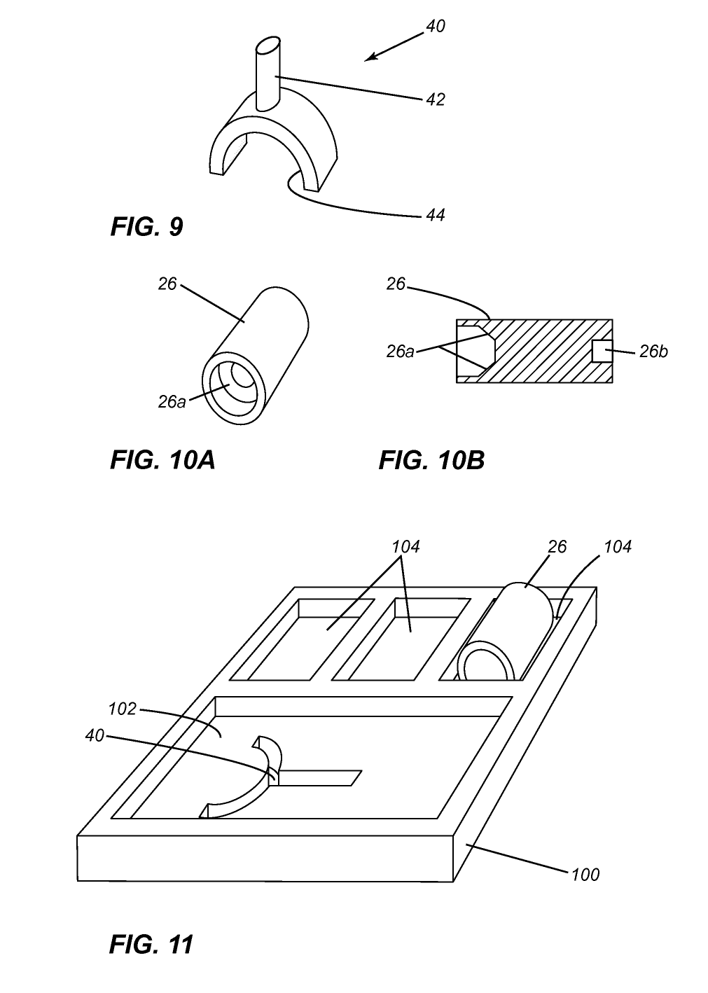

FIG. 9 is an isometric perspective view of the spherometer 40 for use in a surgical instrument tray and/or in a hip resurfacing kit in accordance with embodiments of the present invention. The spherometer has a handle 42 connected to a concave spherical gauge 44 for enabling the surgeon to assess the sphericity of the femoral head.

FIG. 10A is an isometric perspective view of a chamfer reamer 26 (i.e. a tapered reamer) for use in a surgical instrument tray and/or in a hip resurfacing kit in accordance with embodiments of the present invention. FIG. 10B is a cross-sectional view of the chamfered reamer 26 showing that the reamer 26 has a tapered (“chamfered”) cutting surface 26a and a square-profiled (or hexagonally profiled) recess for receiving a square- or hexagonal-profiled rotational driver (such as can be attached on the end of a rotary power tool). The reamer could also be a spherical reamer, or other cutting profile that does not reach the retinacular vessels when applied to the femoral head.

FIG. 11 is an isometric perspective view of a surgical instrument tray 100 having appropriately sized slots, recesses or cavities 102, 104 for accommodating and holding one or more reamers and spherometers. As shown in FIG. 11, surgical instrument tray 100 includes a recess 102 for housing a spherometer 40 and has at least one small recess 104 for accommodating a reamer 26. The number and configuration of slots, recesses and cavities can of course be modified or varied. The surgical instrument tray can be made of stainless steel or other such material, and can be made to be readily sterilizable such as, for example, the multipurpose surgical instrument tray described in U.S. Pat. No. 6,426,041 to Smith, which is hereby incorporated by reference.

In order to implement the method, a surgical instrument tray 100 is provided with both a reaming tool (reamer) 26 and a spherometer 40. In certain operations, the orthopedic surgeon uses both the reamer and the spherometer in a synergistic manner to prepare the femoral head and to restore the sphericity of the femoral head without damaging the retinacular vessels. A surgical instrument tray having both the reamer and the spherometer enables the surgeon to perform this vascular-preserving method in an effective and efficient manner.

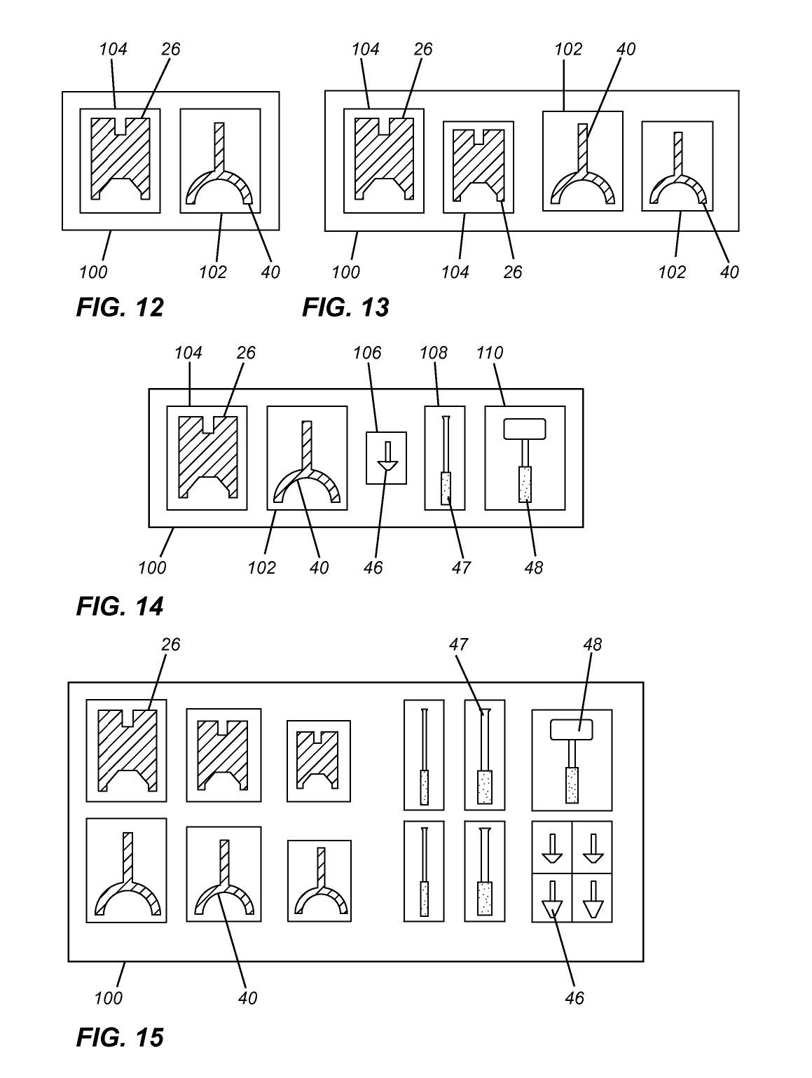

As shown in FIG. 12, the surgical instrument 100 has recesses, slots or cavities 102 and 104 for receiving a reamer 26 and a spherometer 40 (which are shown in cross-section in this and subsequent figures). As shown in FIG. 13, the surgical instrument tray 100 can include multiple reamers 26 and multiple spherometers, for operating on differently sized femoral heads. As shown in FIG. 14, the surgical instrument tray can also include a burr 46, a bone chisel 47 and a mallet 48. Preferably, the surgical instrument tray has appropriately sized recesses or slots 106, 108, and 110 for accommodating the burr, chisel and mallet, respectively.

As shown in FIG. 15, the surgical instrument tray 100 can include multiple (differently sized) reamers 26, multiple (differently sized) spherometers 40, multiple (differently sized) burrs 46, multiple (differently sized) bone chisels 47, and a mallet 48. The surgical instrument trays presented in FIGS. 12 to 15 are meant to be merely illustrative, and therefore, virtually any combination or permutation of instruments can be included provided there is at least one reamer and one spherometer.

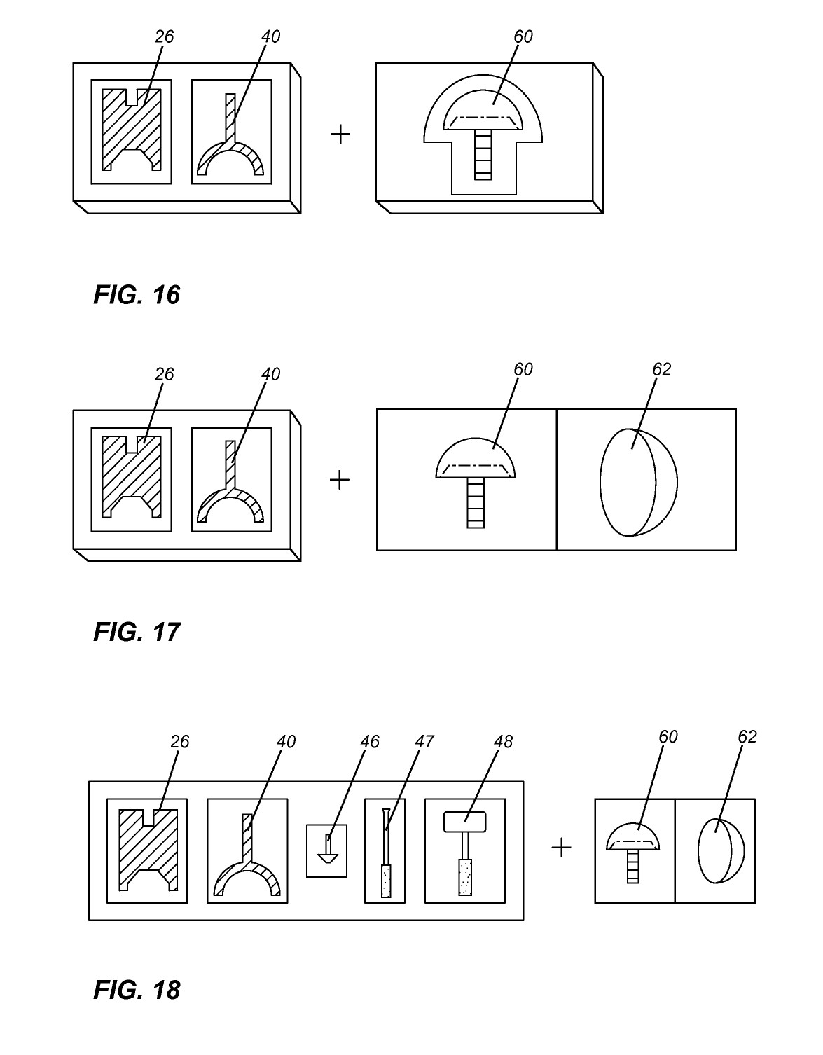

As shown in FIGS. 16 to 18, the surgical instrument tray 100 can be combined with a separately packaged prosthesis (or prostheses) to form a hip resurfacing kit. In other words, the hip resurfacing kit includes a reamer 26, a spherometer 40 and a prosthetic component. For example, one embodiment of the hip resurfacing kit is shown in FIG. 16 where the kit includes a reamer 26, a spherometer 40, and a femoral head prosthesis 60. The kit shown in FIG. 16 would be used for a partial hip resurfacing (i.e. a hemi-arthroplasty). As shown in FIG. 17, another embodiment of the kit includes a reamer 26, a spherometer 40, and a both the femoral head component 60 and the acetabular component 62. The kit shown in FIG. 17 would be used for a total hip resurfacing.

In addition, as shown in FIG. 18, the hip resurfacing kit can include osteophyte-removing tools such a burr 46 and/or a bone chisel 47 (with or without a mallet 48) for enabling an orthopedic surgeon to restore the sphericity the femoral head during the same surgery. Although the hip resurfacing kit shown in FIG. 18 has both the femoral head and acetabular prostheses, it should be understood that a kit with osteophyte-removing tools could be provided with only the femoral head component (or only the acetabular component). Furthermore, it should be understood that while the femoral head component shown in FIGS. 16-18 has threads for fastening to the femur, the femoral head component could be one that does not have threads, i.e. one that is affixed by bone cement, biological fixation (artificial cartilage) or other techniques.

The foregoing method, instrument tray and kit could also be used for biological resurfacing operations, such as, for example, where artificial cartilage (e.g. genetically engineered cartilage) is attached to a femoral head instead of a metal or ceramic prosthesis. In the case of a biological resurfacing, the surgeon would need to restore the sphericity of the femoral head and would possibly also need to prepare the femoral head for receiving the artificial cartilage.

In summary, and without limiting the foregoing, the method, surgical instrument tray and hip resurfacing kit enable an orthopedic surgeon to avoid the retinacular vessels when preparing the femoral head and also when (optionally) restoring the sphericity of the femoral head. As no (or at most very minimal) damage is done to the retinacular vessels, the vascularity of the femoral head is preserved. Since femoral head vascularity is a key factor in bone tissue survival, this method, tray and kit help to enhance the survivability and longevity of prosthetic implants in hip resurfacing operations.

The embodiments of the invention described above are intended to be exemplary only. Persons of ordinary skill in the art of orthopedic surgery will readily appreciate that obvious variations and modifications can be made to the above embodiments which fall within the scope of the invention. The scope of the invention is therefore intended to be limited solely by the scope of the appended claims.

1. A method of resurfacing a femoral head to preserve vascularity in the femoral head, the method comprising steps of:
preparing the femoral head by cutting only a top portion of the femoral head, the top portion of the femoral head being above an anterior lateral portion of the femoral head where the retinacular vessels supply blood to the femoral head; and
securing a femoral head prosthesis to the femoral head.

2. The method as claimed in claim 1 further comprising steps of:
assessing a sphericity of the femoral head to determine whether osteophyte growth on the femoral head is likely to cause femoro acetabular impingement; and
removing osteophytes from the femoral head to restore the sphericity of the femoral head.

3. The method as claimed in claim 1 wherein the step of preparing the femoral head comprises a step of reaming the top portion of the femoral head using a chamfered reamer whereby the femoral head is reamed without damaging the retinacular vessels.

4. The method as claimed in claim 2 wherein the step of preparing the femoral head comprises a step of reaming the top portion of the femoral head using a chamfered reamer whereby the femoral head is reamed without damaging the retinacular vessels.

5. A surgical instrument tray for resurfacing a femoral head while preserving vascularity in the femoral head, the instrument tray comprising:
a spherometer for assessing a sphericity of the femoral head; and
a reamer for preparing the femoral head.

6. The surgical instrument tray as claimed in claim 5 wherein the spherometer comprises:
a handle for holding the spherometer; and
a concave spherical gauge connected to the handle, the concave spherical gauge being adapted to contact an outer surface of the femoral head to enable an orthopedic surgeon to assess the sphericity of the femoral head.

7. The surgical instrument tray as claimed in claim 5 wherein the reamer is a chamfered reamer for preparing only a top portion of the femoral head that is situated above an anterior lateral portion of the femoral head where the retinacular vessels supply blood to the femoral head.

8. The surgical instrument tray as claimed in claim 6 wherein the reamer is a chamfered reamer for preparing only a top portion of the femoral head that is situated above an anterior lateral portion of the femoral head where the retinacular vessels supply blood to the femoral head.

9. The surgical instrument tray as claimed in claim 5 further comprising a tool for removing osteophytes from the femoral head to enable an orthopedic surgeon to restore the sphericity of the femoral head to minimize femoro acetabular impingement when the femoral head rotates relative to the acetabulum, the tool being selected from the group consisting of bone chisels and burrs.

10. The surgical instrument tray as claimed in claim 6 further comprising a tool for removing osteophytes from the femoral head to enable an orthopedic surgeon to restore the sphericity of the femoral head to minimize femoro acetabular impingement when the femoral head rotates relative to the acetabulum, the tool being selected from the group consisting of bone chisels and burrs.

11. The surgical instrument tray as claimed in claim 7 further comprising a tool for removing osteophytes from the femoral head to enable an orthopedic surgeon to restore the sphericity of the femoral head to minimize femoro acetabular impingement when the femoral head rotates relative to the acetabulum, the tool being selected from the group consisting of bone chisels and burrs.

12. The surgical instrument tray as claimed in claim 8 further comprising a tool for removing osteophytes from the femoral head to enable an orthopedic surgeon to restore the sphericity of the femoral head to minimize femoro acetabular impingement when the femoral head rotates relative to the acetabulum, the tool being selected from the group consisting of bone chisels and burrs.

13. A hip resurfacing kit comprising:
a spherometer for assessing a sphericity of the femoral head; and
a reamer for preparing the femoral head to receive the femoral head prosthetic component.

14. The hip resurfacing kit as claimed in claim 13 further comprising a femoral head prosthetic component.

15. The hip resurfacing kit as claimed in claim 14 further comprising an acetabular prosthetic component for attaching to an acetabulum.

16. The hip resurfacing kit as claimed in claim 13 further comprising a tool for removing osteophytes from the femoral head, the tool being selected from the group consisting of bone chisels and burrs.

17. The hip resurfacing kit as claimed in claim 15 further comprising a tool for removing osteophytes from the femoral head, the tool being selected from the group consisting of bone chisels and burrs.

18. The hip resurfacing kit as claimed in claim 13 wherein the reamer is a chamfered reamer for preparing only a top portion of the femoral head that is situated above an anterior lateral portion of the femoral head to thereby avoid damaging the retinacular vessels in the anterior lateral portion of the femoral head.

19. The hip resurfacing kit as claimed in claim 15 wherein the reamer is a chamfered reamer for preparing only a top portion of the femoral head that is situated above an anterior lateral portion of the femoral head to thereby avoid damaging the retinacular vessels in the anterior lateral portion of the femoral head.

20. The hip resurfacing kit as claimed in claim 17 wherein the reamer is a chamfered reamer for preparing only a top portion of the femoral head that is situated above an anterior lateral portion of the femoral head to thereby avoid damaging the retinacular vessels in the anterior lateral portion of the femoral head.
